(19) United States
(12) Patent Application Publication
(10) Pub. No.: US 2012/0130071 A1
(43) Pub. Date: May 24, 2012

(54) METHOD FOR OXIDIZING METHANE

(75) Inventors: Ferdi Schüth, Mulheim an der Ruhr (DE); Regina Palkovits, Aachen (DE); Christian Baltes, Neustadt an der weinstrasse (DE); Markus Antonietti, Potsdam (DE); Arne Thomas, Dallgow Doberitz (DE)

(73) Assignee: STUDIENGESELLSCHAFT KOHLE MBH, Mülheim an der Ruhr (DE)

(21) Appl. No.: 13/386,781

(22) PCT Filed: Jul. 7, 2010
(86) PCT No.: PCT/DE2010/000788

§ 371 Date: Jan. 24, 2012

(30) Foreign Application Priority Data
Jul. 24, 2009 (DE) 10 2009 034 685.6

(51) Int. Cl. C07F 15/00; C07C 305/04

(52) U.S. Cl. 544/181; 558/20

(57) ABSTRACT

Platinum(II) complexes and the use of these complexes for oxidation of methane to methyl sulfate are disclosed, the catalyst having, as ligands, a network based on aromatic N-heterocycles which has at least 2 coordinative nitrogen atoms per platinum atom, and the complex anion is selected from a halide, hydrogensulfate, hydrogensulfite, sulfate, sulfite, methylsulfate, methanol, water, hydroxide, carbon monoxide, hydrogencarbonate and carbonate. The catalyst enables a selective low-temperature oxidation of methane to methyl sulfate or methanol. The catalysts not only exhibit high activity, but can also be removed easily from the reaction mixture and reused over several runs without significant loss of activity.

The present application is a 371 of International Patent Application No. PCT/DE2010/000788, filed Jul. 7, 2010, which claims priority of German Patent Application No. DE 10 2009 034 685.6, filed Jul. 24, 2009, the entire contents of both of which patent applications are incorporated herein by reference.

The present invention relates to a platinum complex and to a process for oxidizing methane to methyl sulfate in the presence of a platinum complex as a catalyst.

The development of catalysts for the direct low-temperature oxidation of methane to methanol is one of the most important challenges in catalysis of the last few decades. The high bond energy of the CH₃—H bond (435 kJ mol⁻¹) and the strong tendency of the system to overoxidation to CO₂ requires not just highly active but also very selective catalysts. Numerous past studies have already addressed this challenge. However, the majority of the catalysts exhibited irreversible bulk metal formation, together with correspondingly low selectivity for methanol. Some very stable Pd, Au and Hg complexes appeared very promising at first, but achieved only very low turnover frequencies (TOFs) of less than 1 h⁻¹. With regard to heterogeneously catalyzed processes, the studies conducted have been virtually exclusively studies well above 250° C. on basic oxides, transition metal oxides and iron complexes encapsulated in zeolites. However, all these catalysts exhibited a great tendency to overoxidation of the methane to CO₂ and only low selectivity for methanol, and so maximum methanol yields of 5% were achievable.

US patent application 2003/0120125 A1 discloses a process for the partial oxidation of lower alkanes with an oxidizing agent and a strong acid in the presence of a catalyst. The catalyst consists of a metal compound from the group of the platinum metals with a heteroatom-containing ligand which can form monodentate or polydentate ligand complexes with the transition metal, with specific mention of platinum-bidiazine complexes. The molecular catalyst is present in dissolved form during the reaction (homogeneous catalysis).

US patent application 2006/0241327 A1 discloses a further process for oxidation of hydro-carbons to the corresponding hydroxyl compound, in which the catalysts used are activated metal complexes of the transition metals Re, Os, Ir, Ru, W and Rh, and the metal is coordinated by an oxidation-resistant ligand. The problem addressed by the invention described in US 2006/0241327 is that of developing a homogeneous catalyst which is present in dissolved form in the reaction.

As already described above, the oxidation of lower alkanes, such as methane, to the corresponding alcohol has several difficulties. It was an object of the present invention to provide a catalyst for the oxidation of methane to methyl sulfate and optional subsequent hydrolysis to methanol, which has a high activity and with which methane can be converted to the corresponding oxidation product with high selectivity in an oxidation reaction. The catalyst should not only have a high activity and selectivity. It was a further object of the present invention to provide a catalyst present in solid form in the reaction solution. The solid catalyst should also have stability over several process cycles.

The present invention accordingly provides a platinum complex which has, as ligand, a network based on aromatic N-heterocycles which has at least 2 coordinative nitrogen atoms per platinum atom.

The inventive platinum complex is based on a ligand system which can be prepared by trimerization and polymerization of aromatic nitriles in the presence of a Lewis acid. The ligands are thermally stable, and are stable even under strongly oxidizing conditions. The platinum complexes are good oxidation catalysts. They are present in solid form and are especially suitable as catalysts in heterogeneous catalysis in liquid and gaseous systems, since they can be removed in a simple manner from the reaction mixture.

### BRIEF DESCRIPTION OF THE DRAWINGS

The invention will now be described in greater detail with reference to the drawings, wherein:

FIG. 1(a-d) constitute a series of micrographs of CTF material;

FIG. 2 constitutes two micrographs of catalyst material; and

FIG. 3 constitutes two graphs showing catalytic various catalysts over several recycling steps.

The inventive platinum complex contains, as ligands, a network obtainable by heating mono-, di-, tri- and/or tetrasubstituted aromatics or heteroaromatics. The polymerization is effected preferably in the presence of a Lewis acid or in a solvent or solvent mixture, preferably in dimethylformamide. The substituents may be selected from cyano, amino, carboxyl, alcohol, boric acid, ether, ester, where, when the aromatic substituent is an ester group, the reactants also contain compounds with amino groups as substituents. Polybenzimidazoles can also be obtained by reaction of the starting materials in the presence of the Lewis acid in a suitable solvent, such as DMF. The network generally forms at high temperatures, and the Lewis acid should therefore be a thermally stable Lewis acid, preferably a metal salt liquid at reaction temperature. ZnCl₂ has been found to be particularly suitable. In an aftertreatment step, the reaction product can be subjected to a thermal treatment, in the course of which partial carbonization of the product takes place. Processes for preparing the ligands are described in P. Kuhn et al., Angew. Chem. Int. Ed., 2008, 47, 3450-3453, A. Thomas et al., J. Mater. Chem., 2008, 18, 4893-4908 and in Thomas et al., Chem. Mater., Vol. 20, 3, 2008.

For preparation of the ligand, suitable compounds have been found to be preferably 1,2-dicyanobenzene, 1,3-dicyanobenzene, 1,4-dicyanobenzene, 1,3,5-tricyanobenzene, 1,2,4,5-tetracyanobenzene, 4,4′-dicyano-biphenyl, 4,4″-dicyanoterphenyl, 2,4-dicyanopyridine, 2,6-dicyanopyridine, tris(4-cyanophenyl)amine, tris(4-cyanophenyl)benzene, tetra(4-cyanophenyl)adamantane, 2,5-dicyanothiophene and 5,5′-dicyanobipyridine and a mixture of 1,1″,2,2″-tetraminobiphenyl and diphenyl 1,3-dicarboxylate or triphenyl 1,3,5-tricarboxylate.

To prepare the ligand, the aromatic or heteroaromatic compound and the Lewis acid are heated generally to a temperature of 100° C. to 800° C., preferably of 250° C. to 750° C. When a metal salt is used as the Lewis acid, the reaction is effected preferably in the melt of this metal salt. The ligand can also be treated by further thermal treatments after the preparation, within the temperature range from 300° C. to 900° C. and preferably at 400° C. to 600° C. In the case of preparation in a solvent or solvent mixture, preferably in dimethylformamide, the aromatic or heteroaromatic compounds are generally heated to temperatures of 50° C. to 300° C., preferably to 150° C. to 200° C. The preparation may also include a thermal treatment of the solid obtained at temperatures between 150° C. and 800° C., preferably between 200° C. and 400° C.

In a preferred embodiment, the ligand is a network which is obtained by heating 1,4-dicyanobenzene in a melt of ZnL₂ within a temperature range between 280° C. and 730° C. An idealized representation of the reaction is given hereinafter, and it is explicitly pointed out that this is an idealized representation of the reaction product, and in fact a multitude of structural elements can be obtained. It is also possible for carbonized products to be present.

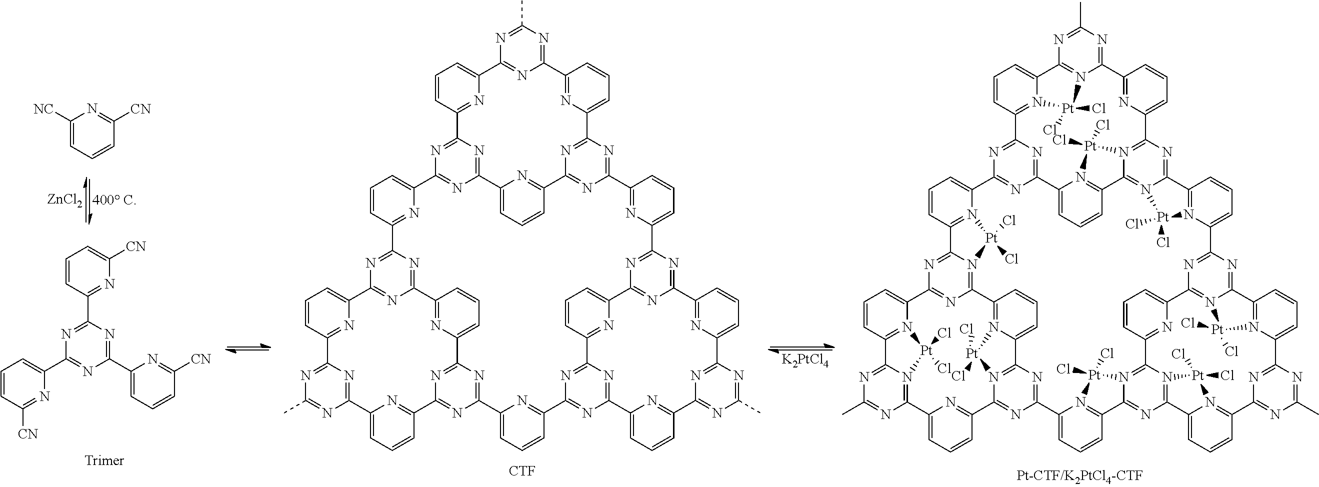

In the inventive platinum complex, the platinum may be in the 0 or +2 oxidation states. Besides the network ligands, it may contain further ligands, such as water, methanol and/or CO. When platinum is in the +2 oxidation state, the complex anion may be selected from any anions which do not adversely affect the reaction. The anion is preferably selected from the group consisting of halide, hydrogensulfate, hydrogensulfite, sulfate, sulfite, methylsulfate, methanol, water, hydroxide, carbon monoxide, hydrogencarbonate and carbonate, and the complex may have water, methanol and/or CO as further ligands.

To prepare the inventive platinum complex, a suitable platinum compound and the ligand material are converted in a manner known per se. In one possible embodiment, a platinum salt, for example an alkali metal tetrahaloplatinate, such as alkali metal₂PtCl₄, is heated in aqueous solution in the presence of the ligand at a temperature between 40° C. and 90° C. over a period of one hour to 10 hours. The reaction product can subsequently be filtered off and dried.

For use as a catalyst, it is also possible to add the starting compounds, i.e. the ligand and the platinum compounds, directly to the reaction mixture; the complex then forms in situ.

The inventive platinum complexes are suitable as catalysts for oxidation of methane to methyl sulfate, which can be hydrolyzed in a subsequent hydrolysis step to methanol.

The present invention further provides a process for oxidizing methane to methyl sulfate in the presence of a platinum complex as a catalyst, which is characterized in that the ligand of the platinum complex is a network based on aromatic N-heterocycles which has at least 2 coordinative nitrogen atoms per platinum atom.

The use of the inventive catalyst enables a low-temperature oxidation of methane to methanol as the end product. The reaction temperature is generally between 100° C. and 300° C., preferably between 160° C. and 250° C.

The oxidizing agents used are sulfuric acid, SO₃ or oleum, preferably in the form of oleum. The reaction proceeds in the individual steps shown below:

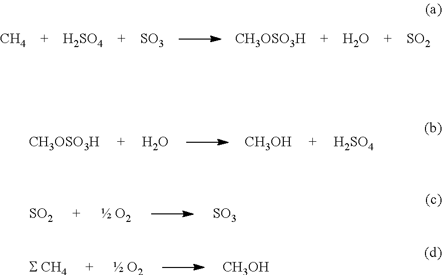

The process according to the invention enables a simple and inexpensive oxidation of methane to methanol. The catalyst can be removed from the reaction mixture in a simple manner, such as by filtration. The oxidation reaction using the inventive catalyst gives, for example, the possibility of using the oxidation process according to the invention for production of natural gas in relatively small deposits from which the natural gas cannot be produced via gas pipes, and of converting it to a transportable material.

### Examples

### Preparation of Ligands

The Pt-bipyrimidine complex and the triazine-based material (CTF) were prepared according to literature methods, J. H. Lunsford, Catal. Today 2000, 63, 165-174 and M. Baerns, J. R. H. Ross in Perspectives in Catalysis (eds.: J. A. Thomas, K. I. Zamaraev), Blackwell, 1992.

### Preparation of the Platinum Complex:

To prepare the platinum complex, 170 mg of CTF and 340 mg of K₂PtCl₄ were converted in water at 60° C. for 4 h, filtered, washed with water and dried at 90° C. overnight.

### Oxidation of Methane

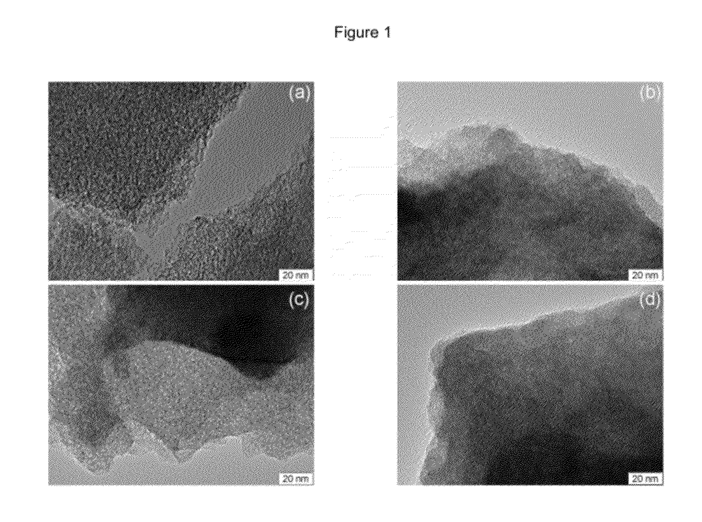

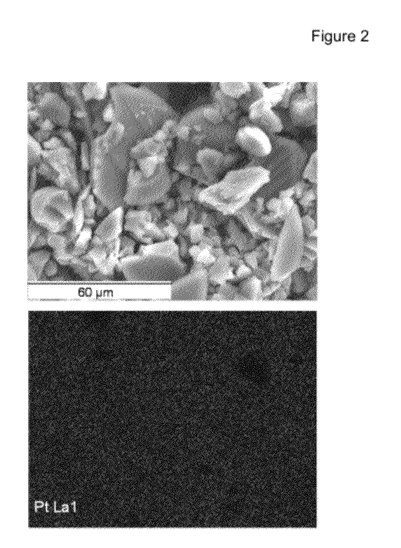

The catalytic tests were conducted in a 50 ml stainless steel autoclave with Teflon insert. In a typical reaction, the autoclave was charged with 15 ml of oleum (30% SO₃) and 50-70 mg of catalyst, sealed and purged repeatedly with argon. In the case of K₂PtCl₄—CTF, 92 mg of CTF and 48 mg of K₂PtCl₄ were added to the oleum in the autoclave. For the actual reaction, the autoclave was pressurized with 40 bar of CH₄, heated up to 215° C., kept there for 2.5 h and then cooled to room temperature. The pressure has to be reduced gradually after the reaction in order to avoid excessive foaming of the reaction solution. In the case of the solid catalysts, the reaction mixture was filtered through a glass frit. The recovered catalyst was washed with a little water in order to remove the predominant portion of remaining sulfuric acid, and dried at 90° C. prior to reuse. The reaction solution was added to 30 ml of water, hydrolyzed at 160° C. under reflux for 4 h and analyzed by means of HPLC. The selectivity for methanol was determined from the pressure drop in the course of the reaction and the FTIR analysis of the gas phase for determination of by-products. In each recycling step of the solid catalyst, about 5-10% by weight of the catalyst material was not recoverable; it remained on the frit. This effect was taken into account in the calculation of catalytic activity. The turnover numbers (TONs) were determined on the basis of the molar ratio of methanol and platinum content of the catalyst as TON=mol_{MeOH}/mol_{Pt}. The platinum content of the catalysts was either determined by means of REM-EDX (Pt—CTF and K₂PtCl₄—CTF, runs 2-6) or the content was estimated on the basis of the amount of platinum used (K₂PtCl₄—CTF, first run). The CTF material was characterized by means of nitrogen sorption, XRD, TEM and XPS analyses, and the catalysts by means of TEM, REM/EDX, XPS and XRD (see FIGS. 1 and 2).

TABLE 1
Catalytic activity of the molecular Periana catalyst and of the solid Pt- and
K₂PtCl₄-CFT catalysts in methane oxidation.

| | Final methanol | |
|---|---|---|
| Catalyst^{[a]} | conc./mol L⁻¹ | TON^{[b]} |
| Catalyst from | 1.65 | 158 |
| U.S. Pat. No. 7,368,598^{[c]} | | |
| Catalyst from | 1.49 | 355 |
| U.S. Pat. No. 7,368,598^{[d]} | | |
| K₂PtCl₄-CTF^{[e]} | 1.54 | 201 |
| Pt-CTF^{[f]} | 1.80 | 246 |

^{[a]}Reaction conditions: 15 ml of H₂SO₄ (30% SO₃), CH₄ pressure 40 bar (25° C.), 2.5 h at 215° C.;
^{[b]}TON based on the platinum content determined from REM-EDX;
^{[c]}65 mg of catalyst;
^{[d]}26 mg of catalyst;
^{[e]}48 mg of CTF with 92 mg of K₂PtCl_{4;}
^{[f]}Results from the second reaction with 62 mg of Pt-CTF.

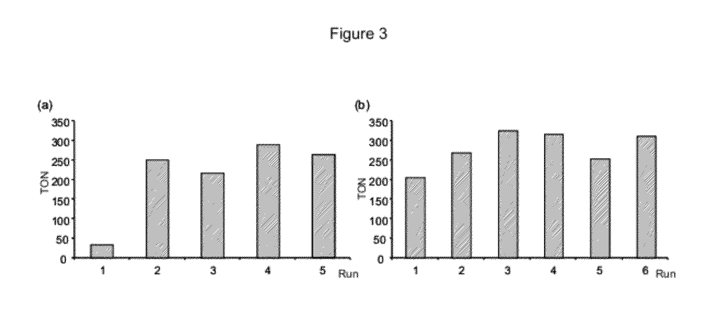

FIG. 3 shows catalytic activity of (a) Pt—CTF and (b) K₂PtCl₄—CFT in the direct oxidation of methane to methanol over several recycling steps.

1. A platinum complex which comprises, as a ligand, a network based on aromatic N-heterocycles which has at least 2 coordinative nitrogen atoms per platinum atom.

2. The platinum complex as claimed in claim 1, wherein the ligand is a network obtainable by heating mono-, di-, tri- or tetrasubstituted aromatics or heteroaromatics, the substituents being selected from cyano, amino, carboxyl, alcohol, boric acid, ether, and ester, where, when the aromatic substituent is an ester group, the reactants also contain compounds with amino groups as substituents, in the presence of a Lewis acid.

3. The platinum complex as claimed in claim 2, wherein the Lewis acid is ZnCl₂.

4. The platinum complex as claimed in claim 2, wherein the substituted aromatic or heteroaromatic is selected from 1,2-dicyanobenzene, 1,3-dicyanobenzene, 1,4-dicyanobenzene, 1,3,5-tricyanobenzene, 1,2,4,5-tetracyanobenzene, 4,4′-dicyano-biphenyl, 4,4″-dicyanoterphenyl, 2,6-dicyanopyridine, 2,4-dicyanopyridine, tris(4-cyanophenyl)amine, tris(4-cyanophenyl)benzene, tetra(4-cyanophenyl)adamantane, 2,5-dicyanothiophene, 5,5′-dicyanobipyridine and a mixture of 1,1′,2,2′-tetraminobiphenyl and diphenyl 1,3-dicarboxylate or triphenyl 1,3,5-tricarboxylate.

5. The platinum complex as claimed in claim 2, wherein the platinum is in the 0 or +2 oxidation state and, when it is in the +2 oxidation state, the complex anion is selected from the group consisting of halide, hydrogensulfate, hydrogensulfite, sulfate, sulfite, methylsulfate, methanol, water, hydroxide, carbon monoxide, hydrogencarbonate and carbonate, and the complex may have water, methanol and/or CO as further ligands.

6. The platinum complex as claimed in claim 2, wherein the substituted aromatic or heteroaromatic is heated in the presence of the Lewis acid to a temperature of 150° C. to 500° C.

7. A process for preparing a platinum complex as claimed in claim 1, comprising heating a platinum compound and the ligand in aqueous solution, wherein the ligand is a network based on aromatic N-heterocycles which has at least 2 coordinative nitrogen atoms per platinum atom.

8. The process as claimed in claim 7, wherein the platinum compound is a dialkali metal tetrahaloplatinate.

9. The process as claimed in claim 7, wherein the solution is heated at a temperature between 40° C. and 90° C. over a period of one hour to 10 hours.

10. A comprising oxidizing methane to methyl sulfate with SO₃ in the presence of a reaction catalyst comprising a platinum complex as claimed in claim 1.

11. A process for oxidizing methane to methyl sulfate in the presence of a platinum complex as a catalyst, wherein the ligand of the platinum complex is a network based on aromatic N-heterocycles which has at least 2 coordinative nitrogen atoms per platinum atom.

12. The process as claimed in claim 11, wherein the catalyst is prepared in situ during the reaction by adding a platinum compound and ligand to the reaction mixture.

13. The process as claimed in claim 11, wherein the methyl sulfate is hydrolyzed to methanol.

14. The process as claimed in claim 11, wherein the oxidizing agent used is H₂SO₄, SO₃ or oleum.
